(19) United States
(12) Patent Application Publication
(10) Pub. No.: US 2022/0033495 A1
(43) Pub. Date: Feb. 3, 2022

(54) ANTIBODY FOR PURE ISOLATION OF VASCULAR ENDOTHELIAL CELLS AND PREPARATION METHOD THEREOF

(71) Applicant: INDUSTRY-ACADEMIC COOPERATION FOUNDATION, YONSEI UNIVERSITY, Seoul (KR)

(72) Inventors: Young Sup YOON, Seoul (KR); Shin Jeong LEE, Seoul (KR); Dong Chan SON, Seoul (KR)

(73) Assignee: INDUSTRY-ACADEMIC COOPERATION FOUNDATION, YONSEI UNIVERSITY, Seoul (KR)

(21) Appl. No.: 17/270,231

(22) PCT Filed: Aug. 20, 2019
(86) PCT No.: PCT/KR2019/010571

§ 371 Date: Feb. 22, 2021

(30) Foreign Application Priority Data
Aug. 20, 2018 (KR) 10-2018-0096650
Aug. 20, 2018 (KR) 10-2018-0096792
Aug. 19, 2019 (KR) 10-2019-0100818
Aug. 19, 2019 (KR) 10-2019-0100831

(51) Int. Cl. C07K 16/28; C12N 5/071; G01N 33/569

(52) U.S. Cl. CPC C07K 16/2803; C07K 16/2896; C12N 2506/45; G01N 33/56966; C12N 5/069

(57) ABSTRACT

The present specification provides an antibody for pure isolation of vascular endothelial cells and a preparation method thereof, the antibody comprising: a heavy chain variable domain and a light chain variable domain which specifically bind to an extracellular matrix domain of PECAM1 having an amino acid sequence of SEQ ID NO: 1; or a heavy chain variable domain and a light chain variable domain which specifically bind to an extracellular matrix domain of CDH5 having an amino acid sequence of SEQ ID NO: 2.

### INCORPORATION-BY-REFERENCE OF SEQUENCE LISTING

The present application is being filed along with a Sequence Listing in electronic format. The Sequence Listing is provided as a file entitled 2021_08_06_INVT-2021137p_SEQLIST.txt, which is 9,749 bytes in size, created and last modified on Aug. 6, 2021. The information in the accompanying Sequence Listing is incorporated by reference in its entirety into this application.

### TECHNICAL FIELD

The present disclosure relates to an antibody for purifying a vascular endothelial cell, and a method for preparation thereof. More specifically, the present disclosure relates to an antibody that is configured to specifically bind to a vascular endothelial cell, and thus may be used for purifying a differentiated vascular endothelial cell, and to a method for preparation thereof.

### BACKGROUND ART

Neovascularization refers to a process by which existing vascular endothelial cells decompose an extracellular matrix (ECM) and migrate, divide, and differentiate to form new capillaries. This neovascularization may be involved in many physiological and pathological phenomena such as wound repair, embryonic development, tumor formation, chronic inflammation, and obesity.

The neovascularization may be an essential phenomenon, especially for wound healing or tissue regeneration. For example, a lack of the neovascularization in a body may lead to dysfunction of tissues or organs as necrosis, ulcers and ischemia occur. Furthermore, poor blood supply may also lead to cardiovascular diseases such as ischemic heart disease, arteriosclerosis, myocardial infarction and angina pectoris. Accordingly, there has been a need for development of new therapies to reduce tissue damage due to the lack of neovascularization and to treat the cardiovascular diseases caused therefrom.

In one example, a new strategy for addressing the lack of neovascularization function, or the cardiovascular disease associated therewith proposes a regenerative therapy as an alternative treatment in which blood vessels are created by inducing the neovascularization from stem cells. An effect of the vascular regeneration treatment using the cell therapy agent may be related to a purity of the cell therapy agent as used, that is, a purity of differentiated vascular endothelial cells. Thus, in order to achieve a desired therapeutic effect, it may be important to separate the endothelial cells of angiogenic ability from various cell lines differentiated from the stem cells at a high purity.

Thus, the development of a purifying method of cell therapeutic agent of vascular endothelial cells to overcome the above limitations and to be used for treatment of deficiency of neovascularization or of the resulting tissue damage, and further, of associated cardiovascular diseases is continuously required.

This “Background Section” has been written to facilitate understanding of the present disclosure. It should not be appreciated that the matters described in the “Background Section” are considered as a prior art.

### DETAILED DESCRIPTION OF THE INVENTION

### Technical Problems

In order to solve the above-mentioned problems, the present inventors continuously studied a method for separating, at a high purity, endothelial cells of angiogenic ability that may be used as cell therapeutic agent, from various cell lines differentiated from stem cells.

As a result, the inventors of the present disclosure were able to find that mRNA and protein expression levels of PECAM1 (platelet endothelial cell adhesion molecule1) and CDH5 (cadherin-5) among markers specifically expressed in differentiated vascular endothelial cells were significantly higher. Furthermore, the inventors of the present disclosure could recognize that sorting PECAM1 or CDH5 expressing positive cells in various cell clusters differentiated from stem cells may allow high-purity vascular endothelial cells to be obtained.

In particular, the inventors of the present disclosure focused on an extracellular matrix domain (ECD) that is present on a surface of the endothelial cells and is configured to connect each endothelial cell to an outside about the PECAM1 protein or the CDH5 protein. More specifically, the inventors of the present disclosure may more easily classify the vascular endothelial cells differentiated from stem cells by developing antibodies capable of binding to the extracellular matrix domain of the PECAM1 protein or the extracellular matrix domain of the CDH5 protein.

As a result, the inventors of the present disclosure developed an antibody capable of binding to the extracellular matrix domain of PECAM1 or the extracellular matrix domain of CDH5. Furthermore, the inventors of the present disclosure could develop a new method for purifying vascular endothelial cells using the same.

Accordingly, the present disclosure aims to provide an antibody for purifying vascular endothelial cells, in which the antibody includes a heavy-chain variable domain and a light-chain variable domain that specifically bind to the extracellular matrix domain of PECAM1 or the extracellular matrix domain of CDH5.

Another purpose of the present disclosure is to further provide an antibody with magnetic particles binding thereto that may more easily purify differentiated vascular endothelial cells via application or blocking of a magnetic force.

Another purpose of the present disclosure is to provide a method for preparing an antibody for purifying vascular endothelial cells, the method including injecting a protein containing an extracellular matrix domain of PECAM1 or an extracellular matrix domain of CDH5 into an antibody producing subject, obtaining a positive clone that reacts with an antigen of PECAM1 or an antigen of CDH5 from the antibody producing subject, and isolating the antibody to PECAM1 or CDH5 from the positive clone.

Another purpose of the present disclosure is to provide a method for purifying vascular endothelial cells, the method including differentiating vascular endothelial cells from stem cells, applying the antibody for purifying the vascular endothelial cell according to an embodiment of the present disclosure to a cell cluster, and classifying cells fluorescing in an immune response using an immunofluorescent staining process as vascular endothelial cells.

Another purpose of the present disclosure is to provide a kit for purifying vascular endothelial cells, the kit including the antibody for purifying vascular endothelial cells according to one embodiment of the present disclosure.

The purposes of the present disclosure are not limited to the above-mentioned purposes, and other purposes not mentioned will be clearly understood by those skilled in the art from the following description.

### Means for Solving the Problems

In order to achieve the above purposes, the present disclosure provides an antibody for purifying vascular endothelial cells, the antibody including a heavy-chain variable domain and a light-chain variable domain that specifically bind to an extracellular matrix domain of PECAM1 having an amino acid sequence represented by SEQ ID NO: 1 or CDH5 having an amino acid sequence represented by SEQ ID NO: 2.

As used herein, the term “endothelial cell” may refer to squamous cells that constitute a layer covering inner walls of a blood vessel and a lymph vessel. Thus, endothelial cells may be used in the same sense as “vascular endothelial cells”.

In one example, in revascularization therapy, endothelial cells differentiated from stem cells, for example, human pluripotent stem cells may be implanted in vivo as cell therapeutic agent to regenerate damaged blood vessels and induce formation or neovascularization of blood vessels. In this connection, the purity of the endothelial cells used for treatment may be associated with prognosis for the vascular regeneration treatment. More specifically, when undifferentiated endothelial cells or endothelial cells containing other cell lines of mesoderm lineages or mixed with impurities are implanted into ischemic tissues, this may lead to deterioration of endothelial cell viability. Thus, as endothelial cells implanted in the regenerative treatment may not contribute to blood vessel formation for a long time, use of low purity endothelial cells may lead to deterioration of the therapeutic effect.

Thus, sorting the high-purity endothelial cells may be associated with not only increasing the yield of the endothelial cells themselves, but also enhancing the effects of cell regeneration treatment using the same.

In one example, the endothelial cell may have a gene or protein that is expressed at a particularly high level. For example, an expression level of PECAM1 gene and CDH5 gene and a level of PECAM1 protein and CDH5 protein in endothelial cells differentiated from stem cells may be higher than in other cell lines differentiated from stem cells. Thus, using PECAM1 and CDH5 as an endothelial cell marker may allow endothelial cells to be separated, at a high purity, from the various differentiated cell lines.

As used herein, the term, “PECAM1” may mean a cell adhesion molecule conjugated to a platelet endothelial cell and may be used interchangeably with CD31, CD31/EndoCAM, PECA1 and endoCAM. In one example, PECAM1 may be found on a surface of platelets, monocytes, neutrophils and some types of T cells. In particular, the PECAM1 may occupy a large portion of an intercellular junction of the endothelial cell. More specifically, the PECAM1 may contain an extracellular matrix domain which exists on the surface of the endothelial cell. In this connection, the extracellular matrix domain of PECAM1 may have more than 80% homology with the amino acid sequence represented by SEQ ID NO: 1, preferably have a homology of at least 90%, and more preferably at least 95% homology with the amino acid sequence represented by SEQ ID NO: 1.

As used herein, the term, “CDH5” may be one of the cadherin family and an intercellular adhesion glycoprotein. In this connection, CDH5 may be used interchangeably with CD144, 7B4, CD144, VE-cadherin (vascular endothelial cadherin) and cadherin 5. In one example, CDH5 may confer homophilic adhesion to endothelial cells and thus may contribute to cohesion and regulation of junctions between endothelial cells. Thus, CDH5 may be associated with maintenance of the barrier of endothelial cells. Furthermore, CDH5 may also be associated with blood vessel development. More specifically, CDH5 may be involved in the maintenance of newly formed blood vessels. In one example, CDH5 may contain an extracellular matrix domain present on the surface of the endothelial cells, and configured to connect each endothelial cell to the outside. In this connection, the extracellular matrix domain of CDH5 may have at least 80% homology with an amino acid sequence represented by SEQ ID NO: 2, preferably at least 90% homology, more preferably at least 95% homology therewith.

Thus, an antibody that specifically binds to the extracellular matrix domain of PECAM1 and CDH5 may be used for purifying vascular endothelial cells.

As used herein, the term “antibody” may mean a glycoprotein produced to fight against the antigen. More specifically, the antibody disclosed herein may be a monoclonal antibody configured to recognize some sequences of the extracellular matrix domain of PECAM1 or the extracellular matrix domain of CDH5 as an epitope and specifically bind thereto. Preferably, the antibody in accordance with the present disclosure may be different from a therapeutic antibody for treatment but is not limited thereto. Further, the antibody in accordance with the present disclosure may preferably be, but is not limited to, a humanized antibody.

In this connection, the antibody may include a heavy-chain variable domain and a light-chain variable domain that specifically bind to the extracellular matrix domain of PECAM1 or the extracellular matrix domain of CDH5.

According to another feature of the present disclosure, the antibody in accordance with the present disclosure may further include a heavy-chain invariable domain and a magnetic particle, in which the magnetic particle may be attached to the heavy-chain invariable domain of the antibody. However, the position of the magnetic particles is not limited thereto. Thus, the vascular endothelial cells detected by the antibody of the present invention including magnetic particles may be more easily purified via the application or blocking of the magnetic force.

In order to achieve the above purpose, the present disclosure provides a method for producing the antibody to purify vascular endothelial cells. More specifically, the method of producing the antibodies for purifying endothelial cell includes injecting a protein containing an extracellular matrix domain of PECAM1 or an extracellular matrix domain of CDH5 into an antibody producing subject so that the antibody against PECAM1 or the antibody against CDH5 are produced, determining a positive clone that reacts with the antigen of PECAM1 or the antigen of CDH5 from the antibody producing subject, and separating the antibody against PECAM1 or the antibody against CDH5 from the positive clone. In this connection, the extracellular matrix domain of PECAM1 has the amino acid sequence represented by SEQ ID NO: 1. Furthermore, the extracellular matrix domain of CDH5 has the amino acid sequence represented by SEQ ID NO: 2.

As used herein, the term “antibody producing subject” may mean any subject that may produce a corresponding antibody to an injected antigen at B lymphocytes thereof. Preferably, the antibody producing subject of the present invention may be a mouse, but is not limited thereto. For example, the antibody producing subject may be a mammal such as a rat, monkey, rabbit, goat, guinea pig, or the like.

As used herein, the term “positive clone” may refer to a clone that produces an antibody that reacts with the antigen of PECAM1 or the antigen of CDH5, more preferably, the extracellular matrix domain of PECAM1 or the extracellular matrix domain of CDH5. For example, clones that exhibit a predetermined level or greater of reactivity with antigens of PECAM1 or CDH5 via an enzyme-linked immune-specific assay (ELISA) may be determined as positive clones.

According to the present disclosure, prior to injecting the antibody producing subject, the method may further include preparing a recombinant plasmid vector containing a base sequence expressing the extracellular matrix domain of PECAM1 or the extracellular matrix domain of CDH5, transfecting the recombinant plasmid vector into a host cell, and obtaining a protein including the extracellular matrix domain of PECAM1 or the extracellular matrix domain of CDH5.

In this connection, the produced protein containing the extracellular matrix domain of the PECAM1 or the CDH5 may be used as an antigen injected into the antibody producing subject for antibody producing.

According to another feature of the present disclosure, determining the positive clone may include isolating a plurality of B lymphocytes from the antibody producing subject, fusing the plurality of B lymphocytes with myeloma cells respectively to produce fused cells, culturing each of the plurality of fused cells to form a clone, and determining the positive clone that reacts with the antigen of PECAM1 or the antigen of CDH5 from the formed clone. Further, separating the antibody may include purifying a monoclonal antibody against PECAM1 or against CDH5 from the positive clone.

As used herein, the term, “B lymphocyte” is a type of lymphocyte that may be responsible for the humoral immune response and may produce antibodies against specific pathogens. In this connection, “B lymphocytes” may be isolated from, but not limited to, spleen cells.

As used herein, the term “fused cell” may refer to a hybrid cell obtained by fusing tumor cells and normal cells having a certain function. In this connection, the fused cells may be used interchangeably with hybridoma and hybrid cells. In one example, the fused cells may be used for producing a monoclonal antibody as they have both the proliferative properties of tumor cells and the functions of living cells.

Antibodies against PECAM1 as obtained according to the method for producing the antibody for purifying the vascular endothelial cells in accordance with the present disclosure may have a concentration of 3.5 mg/ml or greater. In particular, the antibody against PECAM1 as obtained by various examples of the present disclosure may maintain specificity and high bind affinity to the extracellular matrix domain of the PECAM1 at a concentration of 0.002 mg/ml or lower thereof.

Also, antibodies against CDH5 as obtained according to the method for producing the antibody for purifying the vascular endothelial cells in accordance with the present disclosure may have a concentration of 3.5 mg/ml or greater. In particular, the antibody against CDH5 as obtained by various examples of the present disclosure may maintain specificity and high bind affinity to the extracellular matrix domain of the CDH5 at a concentration of 0.002 mg/ml or lower thereof.

In order to achieve the purpose as described above, the present disclosure provides a method for purifying a vascular endothelial cell. More specifically, this method may include differentiating endothelial cells from stem cells to obtain a cell cluster including vascular endothelial cells and endothelial cells different from vascular endothelial cells, applying the antibody for purifying the vascular endothelial cells to the cell cluster so that the protein of CDH5 present on the surface of the vascular endothelial cell and the antibody for purifying the vascular endothelial cell make immune-response, and sorting the cells exhibiting fluorescence in an immunofluorescence staining as vascular endothelial cells.

As used herein, the term “stem cell” may mean, but is not limited to, pluripotent stem cells having the ability to differentiate into various body tissues. Preferably, the stem cells used for the blood vessel regeneration treatment may be human embryonic stem cells or human induced pluripotent stem cells. However, the present disclosure is not limited thereto. For example, the stem cell may be a pluripotent stem cell derived from a mouse or an induced pluripotent stem cell.

As used herein, the term, “cell cluster” may include the inner cell mass of blastocysts, early stage embryos, cord cells, cord blood, human induced pluripotent stem cells, bone marrow, undifferentiated stem cells, stem cells of mesoderm lineage, together with endothelial cells differentiated from stem cells.

As used herein, the term, “immunofluorescence staining” means a method of detecting antigen or antibody present in body fluids and tissues using antibodies or antigens labelled with fluorescent dyes such as fluorescein or rhodamine. In this connection, the immunofluorescence staining method may include direct immunofluorescence staining method using a fluorescent label antibody, indirect immunofluorescence staining method using label-free antibodies that react with antigen and label antibodies that specifically react with label-free antibodies. In one example, the vascular endothelial cells in the cell cluster may fluoresce via the immune response between the antibody to purify the vascular endothelial cells according to one embodiment of the present disclosure and the domain of PECAM1 or the domain of CDH5 present on the cell surface. Thus, detecting the fluorescent signal may allow purifying the vascular endothelial cells.

The purified vascular endothelial cells may be used as cell therapeutic agent. As used herein, the term, “cell therapeutic agent” refers to any drug used for treatment, diagnosis or prevention via a series of actions including the proliferation and selection of viable autologous, allogenic, xenogeneic cells in vitro or changing the biological properties of the cells in order to restore the function of cells and tissues. In accordance with the present disclosure, a cell therapeutic agent may refer to a cell itself that may be transplanted to repair damaged tissue. For example, the cell therapeutic agent may be a vascular endothelial cell differentiated from a human pluripotent stem cell, in which the vascular endothelial cell is implanted in an ischemic site and contributes to blood vessel formation.

In accordance with another feature of the present disclosure, the differentiating may include culturing stem cells in a DLL4 treated medium to differentiate endothelial cells.

As used herein, the term, “DLL4 (delta-like 4)” may be a notch signal delivery ligand. Thus, adding DLL4 to the medium may allow differentiation of the stem cells into endothelial cell lineage cells.

According to another feature of the present disclosure, an antibody for purifying vascular endothelial cells may further contain magnetic particles. Further, the sorting may include permeating a cell cluster to which an antibody to purify vascular endothelial cells is applied into a column receiving metallic particles therein, in which magnetic force is externally applied to the column, and may include sorting cells bound to the metallic particles located inside the column as the vascular endothelial cells.

According to another feature of the present disclosure, the sorting may include sorting the vascular endothelial cells by blocking the magnetic force applied from an outside of the column.

According to the above features, purifying the vascular endothelial cells may be performed more easily using the magnetism.

In order to achieve the above purposes, the present disclosure provides a kit for purifying vascular endothelial cells, the kit including the antibody for purifying vascular endothelial cells according to various embodiments of the present disclosure.

The kit may further include means for measuring the level of immune complexes formed when the antibody for purifying the vascular endothelial cells, and the extracellular matrix domain of PECAM1 or the extracellular matrix domain of CDH5 present on the surface of the vascular endothelial cells are coupled to each other.

According to another feature of the present disclosure, the kit may further include an antigen binding fragment that binds to the epitope sequence of PECAM1 or the epitope sequence of CDH5. Furthermore, the kit may further include a complex of a solid phase and one or more linkers that specifically bind to the antibody or antigen binding fragments.

In this connection, as used herein, the term, “antigen binding fragment” is a fragment of the overall structure of the antibody for purifying the vascular endothelial cells used in the various embodiments of the present disclosure as described above. The antigen binding fragment may mean a polypeptide that may bind to a portion of the extracellular matrix domain of PECAM1 or the extracellular matrix domain of CDH5. For example, the antigen binding fragment may be F (ab′)₂, Fab′, Fab, Fv or scFv. More specifically, the Fab has a structure having a variable domain of the light-chain and heavy-chain, an invariable domain of the light-chain and a first invariable (C_{H1}) of the heavy-chain, and may have a single antigen binding site. The Fab′ may differ from Fab in that the former has a hinge region that includes one or more cysteine residues in a C-terminal of the heavy-chain Cm domain. In one example, F(ab′)₂ antibody may be generated when cysteine residues in the hinge region of Fab′ form disulfide-bond. Fv may be a minimal antibody fragment with only a heavy-chain variable domain and a light-chain variable domain.

As used herein, the term “linker” may refer to a substance that links beads such as magnetic particles and antibody or antigen binding fragments. The linker may be, for example, a nucleic acid, a protein, a polypeptide, a polymer, or a combination thereof, and may have a binding affinity to an antibody or antigen binding fragment. For example, the linker may be Protein A, Protein G, Protein A/G, Protein L, anti-immunoglobulin antibody, Jacalin, or a combination thereof.

As used herein, the term, “solid phase” may mean “mobile phase” in chromatography. In column chromatography, the adsorbent (solid) filling a separation tube or liquid impregnated in a carrier may be the solid phase. The liquid held in a carrier such as a filter paper may be the solid phase in a plate chromatography. Furthermore, in the affinity chromatography, a solid phase may be obtained by binding a compound having a specific affinity to a substance for separating the adsorbent to a solid phase. The solid phase may be, for example, magnetic particles, polystyrene plates or polystyrene beads.

In this connection, the aforementioned linker and solid phase may bind physically or chemically.

According to another embodiment of the present disclosure, the kit may further include a material for separating the solid phase. For example, the kit may further include a buffer or a magnetic force applying substance.

Hereinafter, the present disclosure will be described in more detail with reference to the following examples. However, these examples are only intended to illustrate the present disclosure by way of example. The scope of the present disclosure should not be construed as being limited by these examples.

### Effects of the Invention

The present disclosure provides an antibody that specifically binds to PECAM1 protein or CDH5 protein, thereby purifying only vascular endothelial cells from various cell lines or cell clusters, thereby providing high-purity vascular endothelial cells that may be used as cell therapeutic agent.

In particular, the present disclosure further provides antibodies with magnetic particles bound thereto, thereby more easily purifying differentiated vascular endothelial cells via application or blocking of the magnetic force.

In this connection, the present disclosure provides an antibody for separation, in a purifying manner, of vascular endothelial cells that maintain antigen specificity and binding affinity even at low concentrations thereof, thereby effectively separating the high-purity vascular endothelial cells.

Thus, the vascular endothelial cells isolated using the antibody for purifying the vascular endothelial cells in accordance with the present disclosure may be applied stably clinically as an effective cell therapeutic agent for the prevention or treatment of cardiovascular vessel diseases. More specifically, the present disclosure may provide high-purity vascular endothelial cells that may be applied directly to ischemic tissues, thereby providing the cell therapeutic agent composition for treating diseases that require blood vessel generation, such as ischemic cardiovascular vessel disease, brain blood vessel disease, diabetic complications, and wound care.

Furthermore, the present disclosure provides an antibody for vascular endothelial cell purifying, thereby resulting in solving the problem caused by the low purity of the cell therapeutic agent as used in the conventional blood vessel regeneration treatments, for example, insignificant therapeutic effects due to the lower survival rate in vivo.

The effects of the present disclosure are not limited to the above effects. Rather, more various effects are included in the present specification.

### BRIEF DESCRIPTION OF THE DRAWINGS

FIG. 1A illustrates a procedure of the method of preparation of an antibody against PECAM1 for purifying vascular endothelial cells according to an embodiment of the present disclosure.

FIG. 1B shows the extracellular matrix domains of PECAM1 used as antigens in the preparation method of the antibody against PECAM1 for purifying the vascular endothelial cells according to an embodiment of the present disclosure.

FIG. 1C shows the expression levels for proteins that include extracellular matrix domains of PECAM1 used as antigens in the preparation method of the antibody against PECAM1 for purifying the vascular endothelial cells according to an embodiment of the present disclosure.

FIG. 1D shows the level of binding affinity with antigens as measured for each of the isolated multiple of clones in the preparation method of the antibody against PECAM1 for purifying the vascular endothelial cells according to an embodiment of the present disclosure.

FIG. 1E shows the results of immunofluorescence staining analysis for each of the isolated multiple of clones in the preparation method of the antibody against PECAM1 for purifying the vascular endothelial cells according to an embodiment of the present disclosure.

FIG. 1F illustrates a procedure of the method of preparation of an antibody against CDH5 for purifying vascular endothelial cells according to an embodiment of the present disclosure.

FIG. 1G shows the extracellular matrix domains of CDH5 used as antigens in the preparation method of the antibody against CDH5 for purifying the vascular endothelial cells according to an embodiment of the present disclosure.

FIG. 1H shows the expression levels for proteins that include extracellular matrix domains of CDH5 used as antigens in the preparation method of the antibody against CDH5 for purifying the vascular endothelial cells according to an embodiment of the present disclosure.

FIG. 1I shows the level of binding affinity with antigens as measured for each of the isolated multiple of clones in the preparation method of the antibody against CDH5 for purifying the vascular endothelial cells according to an embodiment of the present disclosure.

FIG. 1J shows the results of immunofluorescence staining analysis for each of the isolated multiple of clones in the preparation method of the antibody against CDH5 for purifying the vascular endothelial cells according to an embodiment of the present disclosure.

FIG. 2A illustrates a procedure of the method of purifying a vascular endothelial cell using an antibody against PECAM1 for purifying the vascular endothelial cell according to another embodiment of the present disclosure.

FIG. 2B illustrates a procedure of the method of purifying a vascular endothelial cell using an antibody against CDH5 for purifying the vascular endothelial cell according to another embodiment of the present disclosure.

FIG. 3A shows the results of immunofluorescence staining analysis for each of the clones determined as multiple of positive clones reacting with PECAM1 in the preparation method of an antibody against PECAM1 for purifying the vascular endothelial cells according to an embodiment of the present disclosure.

FIG. 3B shows the results of immunofluorescence staining for antibodies obtained by the preparation method of the antibody against PECAM1 for purifying the vascular endothelial cells according to an embodiment of the present disclosure.

FIG. 4A and FIG. 4B show the results of evaluation of cell specificity using immunofluorescence staining method for antibodies obtained by the preparation method of the antibody against PECAM1 for purifying the vascular endothelial cells according to an embodiment of the present disclosure.

FIG. 5A shows the results of immunofluorescence staining analysis for each of the clones determined as multiple of positive clones reacting with CDH5 in the preparation method of an antibody against CDH5 for purifying the vascular endothelial cells according to an embodiment of the present disclosure.

FIG. 5B shows the results of evaluation using immunofluorescence staining for antibodies obtained by the preparation method of the antibody against CDH5 for purifying the vascular endothelial cells according to an embodiment of the present disclosure.

FIG. 6A and FIG. 6B show the results of evaluation of cell specificity using immunofluorescence staining process for antibodies obtained by the preparation method of the antibody against CDH5 for purifying the vascular endothelial cells according to an embodiment of the present disclosure.

### BEST MODES FOR CARRYING OUT THE INVENTION

Advantages and features of the present disclosure and methods of achieving them will be apparent with reference to the embodiments described below in detail in conjunction with the accompanying drawings. However, the present disclosure is not limited to the embodiments disclosed below, but may be implemented in various forms. The present embodiments merely allow the disclosure of the present disclosure to be complete and are provided to completely inform the scope of the invention to those of ordinary skill in the technical field to which the present disclosure belongs. The present disclosure is only defined by the scope of the claims.

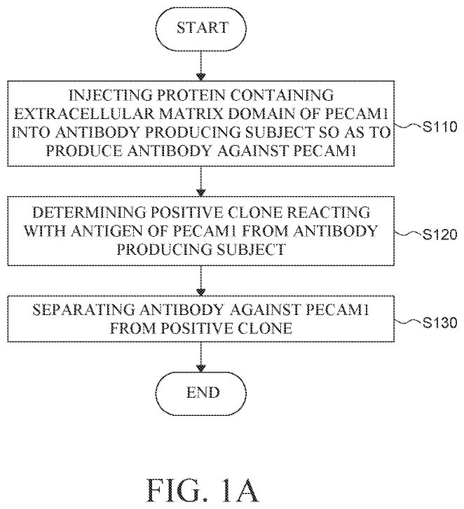

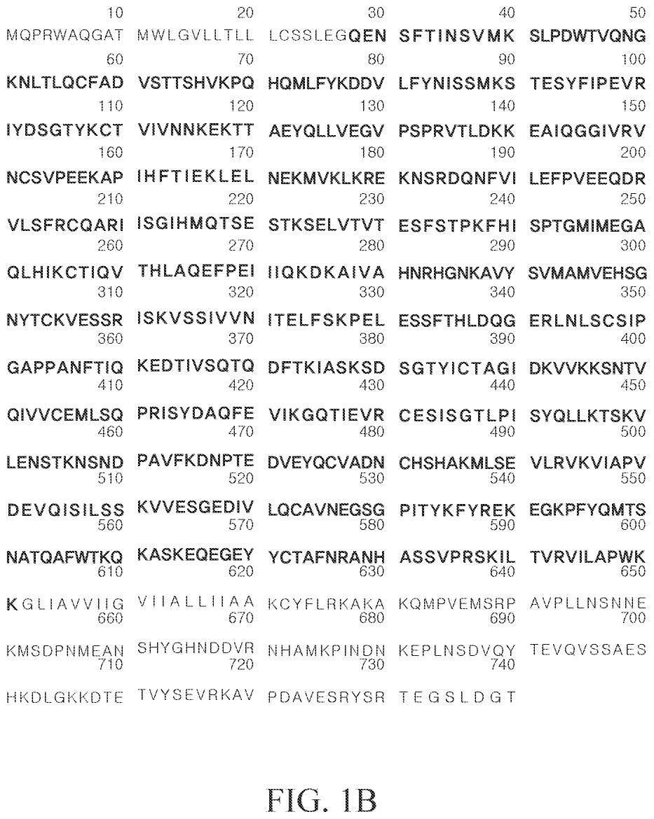

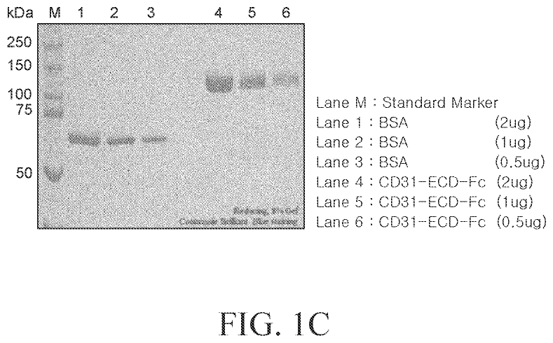

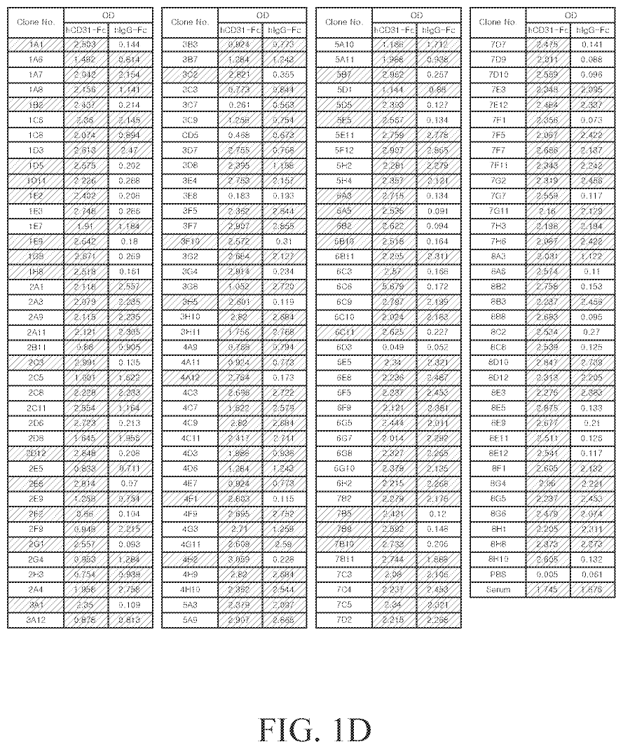

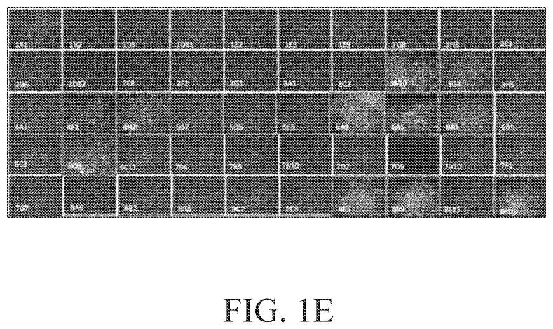

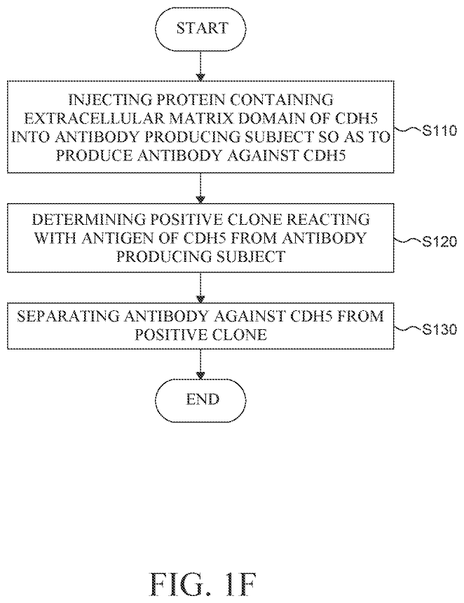

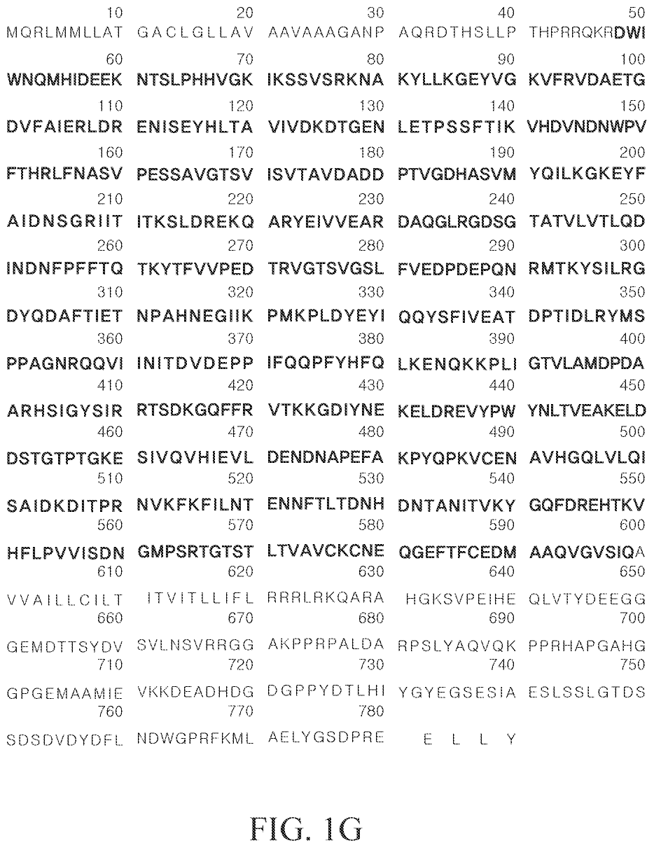

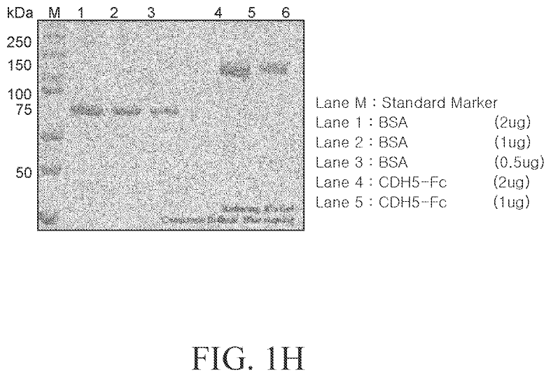

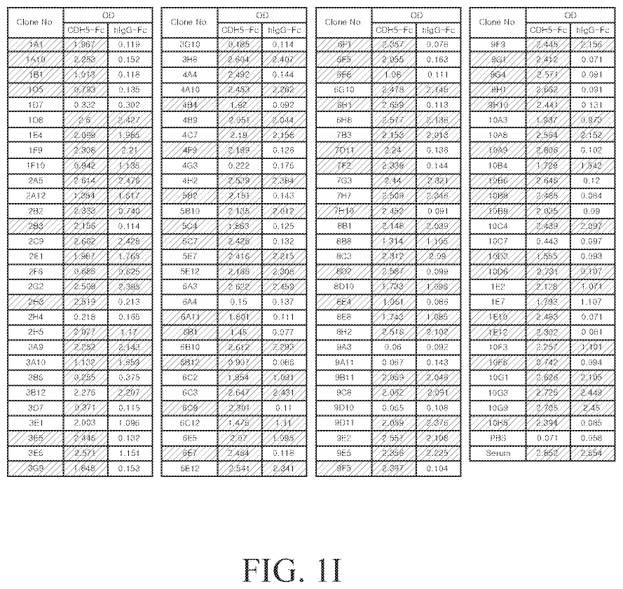

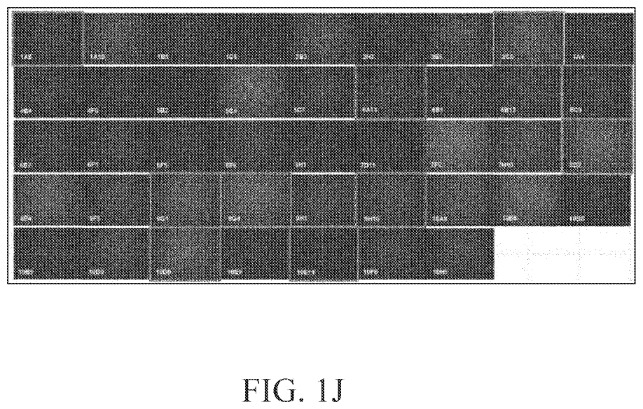

Hereinafter, referring to FIG. 1A to FIG. 1J, a method for preparing an antibodies against PECAM1 and/or CDH5 for purifying vascular endothelial cells will be described in detail. FIG. 1A illustrates a procedure of the method of preparation of an antibody against PECAM1 for purifying vascular endothelial cells according to an embodiment of the present disclosure. FIG. 1B shows the extracellular matrix domains of PECAM1 used as antigens in the preparation method of the antibody against PECAM1 for purifying the vascular endothelial cells according to an embodiment of the present disclosure. FIG. 1C shows the expression levels for proteins that include extracellular matrix domains of PECAM1 used as antigens in the preparation method of the antibody against PECAM1 for purifying the vascular endothelial cells according to an embodiment of the present disclosure. FIG. 1D shows the level of binding affinity with antigens as measured for each of the isolated multiple of clones in the preparation method of the antibody against PECAM1 for purifying the vascular endothelial cells according to an embodiment of the present disclosure. FIG. 1E shows the results of immunofluorescence staining analysis for each of the isolated multiple of clones in the preparation method of the antibody against PECAM1 for purifying the vascular endothelial cells according to an embodiment of the present disclosure. FIG. 1F illustrates a procedure of the method of preparation of an antibody against CDH5 for purifying vascular endothelial cells according to an embodiment of the present disclosure. FIG. 1G shows the extracellular matrix domains of CDH5 used as antigens in the preparation method of the antibody against CDH5 for purifying the vascular endothelial cells according to an embodiment of the present disclosure. FIG. 1H shows the expression levels for proteins that include extracellular matrix domains of CDH5 used as antigens in the preparation method of the antibody against CDH5 for purifying the vascular endothelial cells according to an embodiment of the present disclosure. FIG. 1I shows the level of binding affinity with antigens as measured for each of the isolated multiple of clones in the preparation method of the antibody against CDH5 for purifying the vascular endothelial cells according to an embodiment of the present disclosure. FIG. 1J shows the results of immunofluorescence staining analysis for each of the isolated multiple of clones in the preparation method of the antibody against CDH5 for purifying the vascular endothelial cells according to an embodiment of the present disclosure.

Referring to FIG. 1A, a preparation method of the antibody for purifying vascular endothelial cells according to one embodiment of the present disclosure may include injecting a protein containing an extracellular matrix domain of PECAM1 into the antibody producing subject to produce an antibody against PECAM1 (S110), determining a positive clone to react with the PECAM1 antigen from the antibody producing subject (S120), and separating the antibody against PECAM1 from the positive clone (S130).

More specifically, referring to FIG. 1B, in step S110 of injecting a protein containing an extracellular matrix domain of PECAM1 into an antibody producing subject, a protein having 28th to 681th extracellular matrix domains among the whole protein domain of PECAM1 may be used as an antigen and be injected into an antibody producing subject, for example, a mouse.

In this connection, the protein used as the antigen may be obtained by preparing a recombinant plasmid vector containing a base sequence expressing the extracellular matrix domain of PECAM1, transfecting the recombinant plasmid vector into the host cell, and purifying the protein including the extracellular matrix domain of PECAM1 expressed from the host cell. For example, referring to FIG. 1C, the expression level for the obtained protein of the extracellular matrix domain (ECD) of PECAM1 (CD31) as stained by coomassie blue staining is shown. The purified protein of the extracellular matrix domain of PECAM1 may be used as an antigen injected into the antibody producing subject.

Next, in step S120 of determining a positive clone, it may be obtained a positive clone in which an antibody is formed in an antibody producing subject according to an immune response. For example, referring to FIG. 1D, 51 clones determined as positive clones are shown. The 51 clones are determined as positive clones in serum isolated from mice by ELISA assay. The 51 clones have specificity to the antigen of PECAM1 among the antigens of human PECAM1 (hCD31-Fc) and the human immunoglobulin G antigen (hIgG-Fc), and react with the antigen of the PECAM1 at a high binding affinity. In this connection, the 51 positive clones exhibited high specificity and reactivity with the antigen of human PECAM1. Thus, an antibody against the antigen as the protein of the extracellular matrix domain of PECAM1 may be formed in each of the 51 positive clones.

In one example, the numerically high levels of binding affinity of the antigen-antibody may not mean that the binding affinity with the extracellular matrix domain of PECAM1 on the surface of vascular endothelial cells is substantially high at the cellular level. Therefore, according to another embodiment of the present disclosure, in the step of determining a positive clone (S120), the immunofluorescence staining method may further determine the positive clone including the antibody actually having high binding affinity with the extracellular matrix domain of PECAM1 in the cell. For example, referring to FIG. 1E, the results of performing the immunofluorescence staining method on human umbilical vein endothelial cells (HUVEC) as a human vascular endothelial cell in order to select a clone that can actually accurately detect the position of PECAM1 expression in vascular endothelial cells among the 51 clones as determined as positive clones via ELISA are shown. In this connection, among the 51 positive clones, 3F10, 4F1, 4H2, 6A3, 6A5, 6B2, 6C6, 8E5, 8E9 and 8H10 appear to fluoresce more strongly than other clones. In other words, clones of 3F10, 4F1, 4H2, 6A3, 6A5, 6B2, 6C6, 8E5, 8E9 and 8H10 were found to actually have high binding affinity with the extracellular matrix domain of PECAM1 in the human vascular endothelial cell and thus, finally, may be determined as a highly reactive positive clone.

According to another embodiment of the present disclosure, the step of determining a positive clone (S120) may include isolating a plurality of B lymphocytes from the antibody producing subject, fusing the plurality of B lymphocytes with myeloma cells respectively to produce fused cells, culturing each of the plurality of fused cells to form a clone, and determining the positive clone that reacts with the antigen of PECAM1 from the formed clone.

Finally, separating the antibody against PECAM1 (S130) may include culturing positive clones in large quantities, purifying antibodies against PECAM1, more specifically humanized PECAM1 monoclonal antibodies from the clones, thereby to obtain antibodies for purifying vascular endothelial cells.

According to another embodiment of the present disclosure, the step of isolating the antibody against PECAM1 (S130) may include passing the positive clone obtained in the step of determining the positive clone (S120) through an equilibrated column to remove the secondary proteins, and passing the protein-removed positive clone through the PECAM1 antigen-coated column to purify PECAM1 antigen-specific antibodies that may be used as antibodies for purifying the vascular endothelial cells in accordance with the present disclosure.

The PECAM1 antigen-specific antibodies as obtained by the above method may be directly applied to, in particular, ischemic tissues. Alternatively, the PECAM1 antigen-specific antibodies may be used for purifying vascular endothelial cells used in the cell therapeutic agent that may be used for the treatment of such diseases.

According to another embodiment of the present disclosure, the antibody against CDH5 for purifying vascular endothelial cells can be produced by flowing steps.

Referring to FIG. 1F, a preparation method of the antibody for purifying vascular endothelial cells according to one embodiment of the present disclosure may include injecting a protein containing an extracellular matrix domain of CDH5 into the antibody producing subject to produce an antibody against CDH5 (S110), determining a positive clone to react with the CDH5 antigen from the antibody producing subject (S120), and separating the antibody against CDH5 from the positive clone (S130).

More specifically, referring to FIG. 1G, in step S110 of injecting a protein containing an extracellular matrix domain of CDH5 into an antibody producing subject, a protein having 48th to 599th extracellular matrix domains among the whole protein domain of CDH5 may be used as an antigen and be injected into an antibody producing subject, for example, a mouse.

In this connection, the protein used as the antigen may be obtained by preparing a recombinant plasmid vector containing a base sequence expressing the extracellular matrix domain of CDH5, transfecting the recombinant plasmid vector into the host cell, and purifying the protein including the extracellular matrix domain of CDH5 expressed from the host cell. For example, referring to FIG. 1H, the expression level for the obtained protein of the extracellular matrix domain (ECD) of CDH5 as stained by a coomassie blue staining is shown. The purified protein of the extracellular matrix domain of CDH5 may be used as an antigen injected into the antibody producing subject.

Next, in step S120 of determining a positive clone, it may be obtained a positive clone in which an antibody is formed in an antibody producing subject according to an immune response. For example, referring to FIG. 1I, 43 clones determined as positive clones are shown. The 43 clones are determined as positive clones in serum isolated from mice by ELISA assay. The 43 clones have specificity to the antigen of CDH5 among the antigens of human CDH5 (hCDH5-Fc) and the human immunoglobulin G antigen (hIgG-Fc), and react with the antigen of the CDH5 at a high binding affinity. In this connection, antigen of human immunoglobulin G was used as a control antigen. The 43 positive clones exhibited high specificity and reactivity with the antigen of human CDH5 in contrast to the immunoglobulin G antigen of the control group. Thus, an antibody against the antigen as the protein of the extracellular matrix domain of CDH5 may be formed in each of the 43 positive clones. More specifically, the greater the difference between the OD value measured for the antigen of immunoglobulin G and the OD value measured for the antigen of human CDH5, the higher the binding affinity of the antigen-antibody.

In one example, the numerically high levels of binding affinity of the antigen-antibody may not mean that the binding affinity with the extracellular matrix domain of CDH5 present on the surface of vascular endothelial cells is substantially high at the cellular level. Therefore, according to another embodiment of the present disclosure, in the step of determining a positive clone (S120), the immunofluorescence staining method may further determine the positive clone including the antibody actually having high binding affinity with the extracellular matrix domain of CDH5 in the cell. For example, referring to FIG. 1J, the results of performing the immunofluorescence staining method on human umbilical vein endothelial cells (HUVEC) as a human vascular endothelial cell in order to select a clone that can actually accurately detect the position of CDH5 expression in vascular endothelial cells among the 43 clones as determined as positive clones via ELISA are shown. In this connection, among the 43 positive clones, 1A5, 3G9, 6A11, 6C9, 8D2, 9G1, 9G4, 9H10, 10D6 and 10E11 appear to fluoresce more strongly than other clones. In other words, clones of 1A5, 3G9, 6A11, 6C9, 8D2, 9G1, 9G4, 9H10, 10D6 and 10E11 were found to actually have high binding affinity with the extracellular matrix domain of CDH5 in the human vascular endothelial cell and thus, finally, may be determined as a highly relative positive clone.

According to another embodiment of the present disclosure, the step of determining a positive clone (S120) may include isolating a plurality of B lymphocytes from the antibody producing subject, fusing a plurality of B lymphocytes with myeloma cells respectively to produce fused cells, culturing each of the plurality of fused cells to form a clone, and determining the positive clone that reacts with the antigen of CDH5 from the formed clone.

Finally, separating the antibody against CDH5 (S130) may culturing positive clones in large quantities, purifying antibodies against CDH5, more specifically humanized CDH5 monoclonal antibodies from the clones, thereby to obtain antibodies for purifying vascular endothelial cells.

According to another embodiment of the present disclosure, the step of isolating the antibody to CDH5 (S130) may include passing the positive clone obtained in the step of determining the positive clone (S120) through an equilibrated column to remove the secondary proteins, and passing the protein-removed positive clone through the CDH5 antigen-coated column to purify CDH5 antigen specific antibodies that may be used as antibodies for purifying the vascular endothelial cells in accordance with the present disclosure.

The CDH5 antigen-specific antibodies as obtained by the above method may be directly applied to, in particular, ischemic tissues. Alternatively, the CDH5 antigen-specific antibodies may be used for purifying vascular endothelial cells used in the cell therapeutic agent that may be used for the treatment of such diseases.

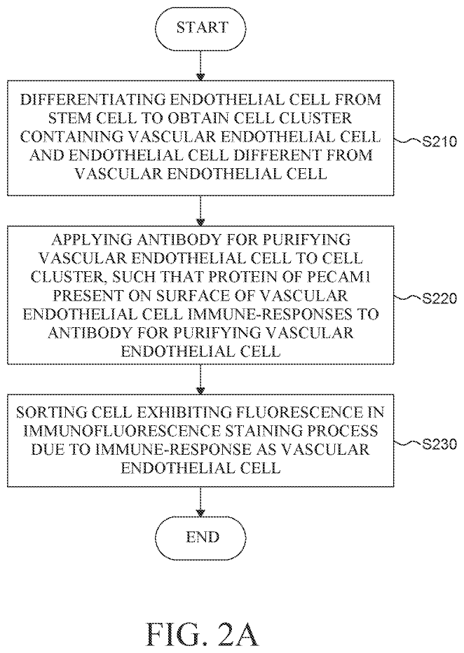

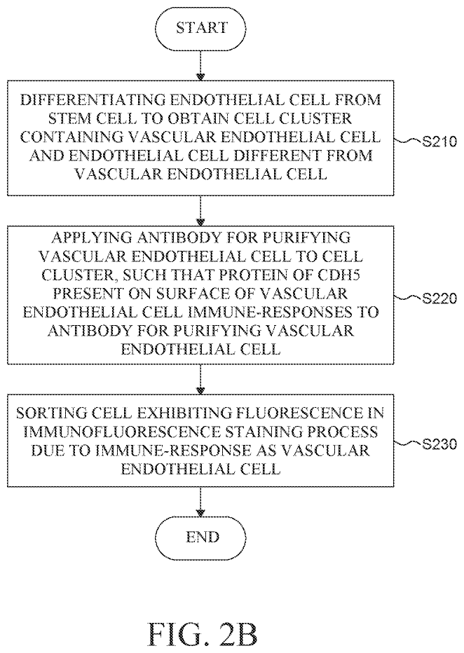

Hereinafter, referring to FIG. 2A and FIG. 2B, a method for purifying a vascular endothelial cell using an antibody against PECAM1 or against CDH5 for purifying a vascular endothelial cell according to an embodiment of the present disclosure will be described in detail. FIG. 2A illustrates a procedure of the method of purifying a vascular endothelial cell using an antibody against PECAM1 for purifying the vascular endothelial cell according to another embodiment of the present disclosure. FIG. 2B illustrates a procedure of the method of purifying a vascular endothelial cell using an antibody against CDH5 for purifying the vascular endothelial cell according to another embodiment of the present disclosure.

Referring to FIG. 2A, the purifying method of the vascular endothelial cell according to another embodiment of the present disclosure may include differentiating the endothelial cells from the stem cells to obtain a cell cluster including the vascular endothelial cells (S210), applying the antibody for purifying the vascular endothelial cells to the cell cluster (S220), and sorting the vascular endothelial cells based on the level of fluorescence according to the immunofluorescence staining method (S230).

According to another embodiment of the present disclosure, in the step of differentiating the endothelial cell (S210), a human pluripotent stem cell or a human induced pluripotent stem cell may be used as the stem cell. Furthermore, differentiation of endothelial cells can be initiated by culturing the stem cells in a medium treated with DLL4 (notch signaling ligand).

According to another embodiment of the present disclosure, the cell cluster obtained in the step of differentiating the endothelial cells (S210) may include the inner cell mass of blastocysts, early stage embryos, cord cells, cord blood, human induced pluripotent stem cells, bone marrow, undifferentiated stem cells, stem cells of mesoderm lineage, together with endothelial cells differentiated from stem cells. However, the present is not limited thereto.

According to another embodiment of the present disclosure, the antibody used for purifying a vascular endothelial cell may further include magnetic particles. Accordingly, the step of sorting (S230) may include permeating a cell cluster to which an antibody for purifying vascular endothelial cells is applied into a column receiving metallic particles therein, in which magnetic force is externally applied to the column, and may include sorting cells bound to the metallic particles located inside the column as the vascular endothelial cells.

According to another feature of the present disclosure, the step of sorting (S230) may include sorting the vascular endothelial cells by blocking the magnetic force applied from an outside of the column.

The vascular endothelial cells isolated, at the high purity, in this way are directly used for treatment of ischemic tissues, or may be used as a cell therapeutic agent for treatment of secondary induced diseases such as cardiovascular vessel diseases such as ischemic heart disease, arteriosclerosis, myocardial infarction and angina pectoris.

Referring to FIG. 2B more, the purifying method of the vascular endothelial cell according to another embodiment of the present disclosure may include differentiating the endothelial cells from the stem cells to obtain a cell cluster including the vascular endothelial cells (S210), applying the antibody for purifying the vascular endothelial cells to the cell cluster (S220), and sorting the vascular endothelial cells based on the level of fluorescence according to the immunofluorescence staining method (S230).

According to another embodiment of the present disclosure, in the step of differentiating the endothelial cell (S210), a human pluripotent stem cell or a human induced pluripotent stem cell may be used as the stem cell. Furthermore, differentiation of endothelial cells can be initiated by culturing the stem cells in a medium treated with DLL4 (notch signaling ligand).

According to another embodiment of the present disclosure, the cell cluster obtained in the step of differentiating the endothelial cells (S210) may include the inner cell mass of blastocysts, early stage embryos, cord cells, cord blood, human induced pluripotent stem cells, bone marrow, undifferentiated stem cells, stem cells of mesoderm lineage, together with endothelial cells differentiated from stem cells. However, the present is not limited thereto.

According to another embodiment of the present disclosure, the antibody used for purifying a vascular endothelial cell may further include magnetic particles. Accordingly, the sorting step (S230) may include permeating a cell cluster to which an antibody to purify vascular endothelial cells is applied into a column receiving metallic particles therein, wherein magnetic force is externally applied to the column, and may include sorting cells bound to the metallic particles located inside the column as the vascular endothelial cells.

According to another feature of the present disclosure, the sorting (S230) may further include sorting the vascular endothelial cells by blocking the magnetic force applied from an outside of the column.

The vascular endothelial cells isolated, at the high purity, in this way are directly used for treatment of ischemic tissues, or may be used as a cell therapeutic agent for treatment of secondary induced diseases such as cardiovascular vessel diseases such as ischemic heart disease, arteriosclerosis, myocardial infarction and angina pectoris.

### Example 1: Detection of Vascular Endothelial Cells Using Antibodies to Purify Vascular Endothelial Cells According to Embodiment of Present Disclosure Antibody Against PECAM1

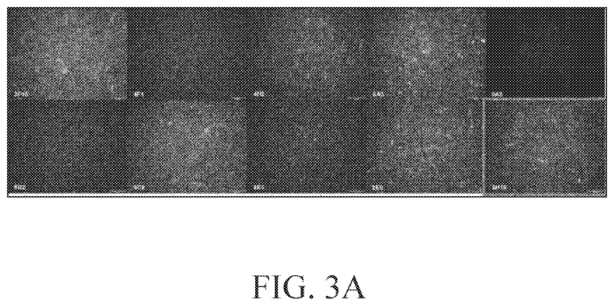

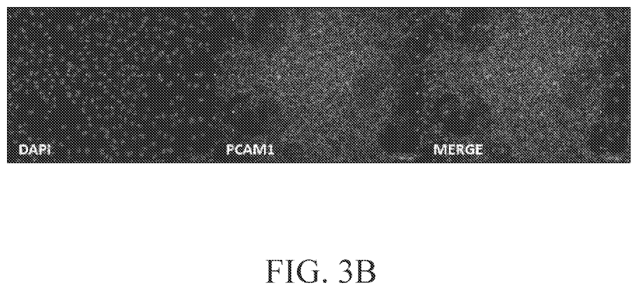

Hereinafter, referring to FIG. 3A and FIG. 3B, the evaluation results of the antibody for purifying the vascular endothelial cells according to an embodiment of the present disclosure will be described. In this connection, HUVEC cells, that is, human vascular endothelial cells were used as vascular endothelial cells. However, the effect of the antibodies to purify vascular endothelial cells in accordance with the present disclosure is not limited thereto. For example, the antibodies to purify vascular endothelial cells may have the same effect on a mouse vascular endothelial cell.

FIG. 3A shows the results of immunofluorescence staining analysis for each of the clones determined as multiple of positive clones reacting with PECAM1 in the preparation method of an antibody against PECAM1 for purifying the vascular endothelial cells according to an embodiment of the present disclosure. FIG. 3B shows the results of immunofluorescence staining for antibodies obtained by the preparation method of the antibody against PECAM1 for purifying the vascular endothelial cells according to an embodiment of the present disclosure.

Referring to FIG. 3A, there are shown results of detection of vascular endothelial cells when using antibodies purified from 10 positive clones 3F10, 4F1, 4H2, 6A3, 6A5, 6B2, 6C6, 8E5, 8E9 and 8H10 as eventually determined as positive clones in the producing method of the antibody for purifying vascular endothelial cells according to an embodiment of the present disclosure. More specifically, the antibodies of 3F10, 4H2, 6A3, 6C6, 8E9 and 8H10 are specific to the extracellular matrix domain of PECAM1 present on the surface of vascular endothelial cells and thus detect the vascular endothelial cells in a strong signaling manner. In particular, the antibody of 8H10 detects PECAM1 with a stronger signal than other antibodies. Referring to FIG. 3B, there are shown results of detection of vascular endothelial cells when using the antibody of 8H10. The antibody of 8H10 may be obtained by producing antibodies from a mouse using the 8H10 clones that are highly reactive with vascular endothelial cells, and, then, by purifying the antibodies. In this connection, the concentration of the obtained antibody of 8H10 was 3.86 mg/ml. The content of the antibodies of 8H10 was diluted at a 1000:1 ratio to perform an immunofluorescence staining method for HUVEC cells. More specifically, the endothelial cells detected by the reaction of the antibody of 8H10 with the PECAM1 protein present on the cell surface were generally consistent with the vascular endothelial cells stained by DAPI (4′,6′-diamidine-2′-phenylindole dihydrochloride). That is, the antibody of the 8H10 may effectively detect the vascular endothelial cells and may be used for purifying vascular endothelial cells.

In particular, the antibody of 8H10 has specificity to vascular endothelial cells and has a high degree of binding to the vascular endothelial cells at a concentration of about 0.00386 mg/ml following the dilution at 1000:1.

From a result of Example 1, the antibody for purifying the vascular endothelial cells used in various embodiments of the present disclosure may include antibodies of 3F10, 4F1, 4H2, 6A3, 6A5, 6B2, 6C6, 8E5, 8E9 and 8H10, preferably, antibodies of 3F10, 4H2, 6A3, 6C6, 8E9 and 8H10, more preferably, the antibody of 8H10. However, the present invention is not limited thereto.

### Example 2: Evaluation of Cell Specificity of Antibodies for Purifying Vascular Endothelial Cells According to Embodiment of Present Disclosure Antibody Against PECAM1

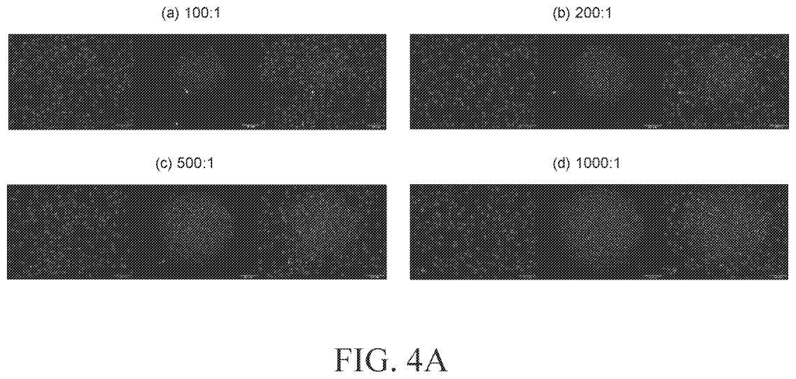

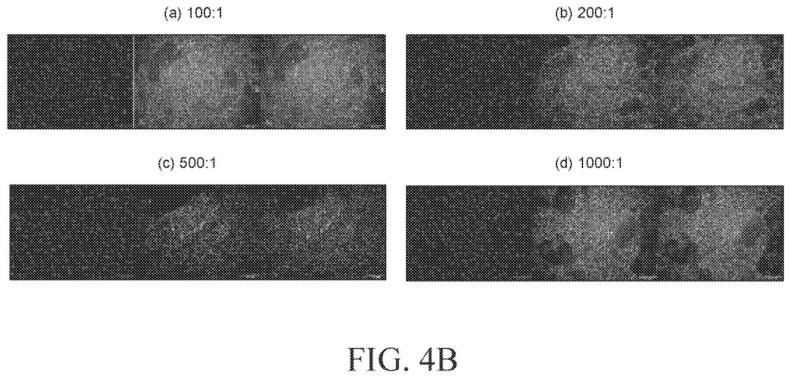

Hereinafter, referring to FIG. 4A and FIG. 4B, the results of evaluation of cell specificity of antibodies for purifying vascular endothelial cells according to an embodiment of the present disclosure will be described. In this connection, HUVEC cells as human vascular endothelial cells were used as vascular endothelial cells. As a negative control, fibroblasts were used. In this connection, the content of the antibodies in accordance with the present disclosure, that is, 3.86 mg/ml concentration of the antibodies of 8H10 as described above in Example 1 was diluted in each of ratios of 100:1, 200:1, 500:1 and 1000:1. Then, the diluted antibodies were applied to fibroblast cells and human vascular endothelial cells, respectively.

FIG. 4A and FIG. 4B show the results of evaluation of cell specificity using immunofluorescence staining method for antibodies obtained by the preparation method of the antibody against PECAM1 for purifying the vascular endothelial cells according to an embodiment of the present disclosure.

First, referring to (a), (b), (c) and (d) of FIG. 4A, the results of the application of the antibodies in accordance with the present disclosure as diluted in ratios of 100:1, 200:1, 500:1 and 1000:1 to the fibroblasts as the negative control are shown. More specifically, it is observed that fibroblasts emit blue light. However, as the fibroblasts have no specific binding to the antibody in accordance with the present disclosure, the antibody emitting green light is not observed.

In one example, referring to (a), (b), (c) and (d) of FIG. 4B, together with blue luminescent human vascular endothelial cells, a green luminescent antibody appears on the surface of the endothelial cell. In this connection, the antibody in accordance with the present disclosure specifically binds to PECAM1 on the surface of human vascular endothelial cells, allowing the detection of the human vascular endothelial cells. In particular, referring to (d) of FIG. 4B, the antibody in accordance with the present disclosure at a diluted state at 1000:1 allows a high level of detection of vascular endothelial cells. Thus, the antibody in accordance with the present disclosure has a high specificity to the vascular endothelial cells even at low concentrations. Even at low concentrations of the antibodies, the vascular endothelial cells may be detected efficiently. Furthermore, the antibody in accordance with the present disclosure may facilitate the acquisition of the endothelial cells at the high purity.

In particular, the antibody of 8H10 has the specificity to the vascular endothelial cells and has a high degree of binding to the vascular endothelial cells at a concentration of about 0.00386 mg/ml following a dilution at 1000:1.

From a result of Example 2, the antibody for purifying vascular endothelial cells, which are used in various embodiments of the present disclosure may be used to isolate high purity endothelial cells as the antibody binds specifically to the vascular endothelial cells.

Thus, the vascular endothelial cells isolated using the antibody for purifying the vascular endothelial cells in accordance with the present disclosure may have a high purity and thus may be stably clinically applied as an effective cell therapy for the prevention or treatment of cardiovascular vessel diseases.

More specifically, the antibodies used in the various embodiments of the present disclosure may purify, in a high purity, the vascular endothelial cells that may be applied directly to ischemic tissue. Thus, the antibodies may constitute a cell therapeutic composition for treatment of diseases requiring blood vessel generation, such as ischemic heart blood vessel disease, brain blood vessel disease, diabetic complications, and wound care.

### Example 3: Detection of Vascular Endothelial Cells Using Antibodies to Purify Vascular Endothelial Cells According to Embodiment of Present Disclosure Antibody Against CDH5

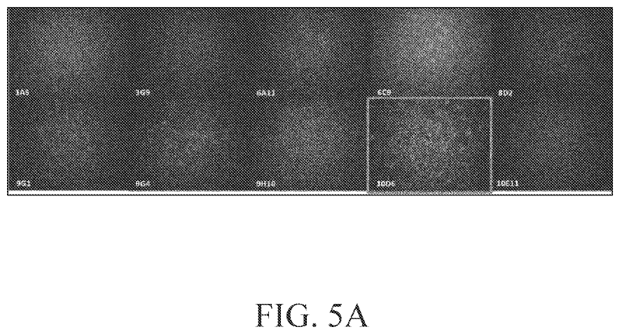

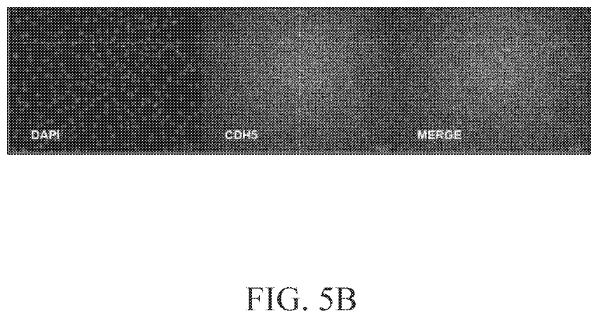

Hereinafter, referring to FIG. 5A and FIG. 5B, the evaluation results of the antibody for purifying the vascular endothelial cells according to an embodiment of the present disclosure will be described. In this connection, HUVEC cells, that is, human vascular endothelial cells were used as vascular endothelial cells. However, the effect of the antibodies to purify vascular endothelial cells in accordance with the present disclosure is not limited thereto. For example, the antibodies to purify vascular endothelial cells may have the same effect on a mouse vascular endothelial cell.

FIG. 5A shows the results of immunofluorescence staining analysis for each of the clones determined as multiple of positive clones reacting with CDH5 in the preparation method of an antibody against CDH5 for purifying the vascular endothelial cells according to an embodiment of the present disclosure. FIG. 5B shows the results of evaluation using immunofluorescence staining for antibodies obtained by the preparation method of the antibody against CDH5 for purifying the vascular endothelial cells according to an embodiment of the present disclosure.

Referring to FIG. 5A, there are shown results of detection of vascular endothelial cells when using antibodies purified from 10 clones 1A5, 3G9, 6A11, 6C9, 8D2, 9G1, 9G4, 9H10, 10D6 and 10E11 eventually determined as the positive clones in the producing method of the antibody for purifying vascular endothelial cells according to one embodiment of the present disclosure. More specifically, the antibodies of 3F10, 4H2, 6A3, 6C6, 8E9 and 8H10 are specific to the extracellular matrix domain of CDH5 present on the surface of vascular endothelial cells and thus detect the vascular endothelial cells in a strong signaling manner. In particular, the antibody of 10D6 detects CDH5 with a stronger signal than other antibodies.

Referring to FIG. 5B, there are shown results of detection of vascular endothelial cells using the antibody of 10D6. The antibody of 10D6 may be obtained by producing antibodies from a mouse using the 10D6 clones that are highly reactive with vascular endothelial cells, and, then, by purifying the antibodies. In this connection, the concentration of the obtained antibody of 10D6 was 3.86 mg/ml. The content of the antibodies of 10D6 was diluted at a 1000:1 ratio to perform an immunofluorescence staining method for HUVEC cells. More specifically, the endothelial cells detected by the reaction of the antibody of 10D6 with the CDH5 protein present on the cell surface were generally consistent with the vascular endothelial cells stained by DAPI (4′,6′-diamidine-2′-phenylindole dihydrochloride). That is, the antibody of the 10D6 may effectively detect the vascular endothelial cells and may be used for purifying vascular endothelial cells.

In particular, the antibody of 10D6 has specificity to vascular endothelial cells and has a high degree of binding to the vascular endothelial cells at a concentration of about 0.00386 mg/ml following the dilution at 1000:1.

From a result of Example 3, the antibody for purifying the vascular endothelial cells used in various embodiments of the present disclosure may include antibodies of 1A5, 3G9, 6A11, 6C9, 8D2, 9G1, 9G4, 9H10, 10D6 and 10E11, preferably, antibodies of 1A5, 6A11, 6C9, 9G1, 9G4, 9H10 and 10D6, more preferably, the antibody of 10D6. However, the present invention is not limited thereto.

### Example 4: Evaluation of Cell Specificity of Antibodies for Purifying Vascular Endothelial Cells According to Example of Present Disclosure Antibody Against CDH5

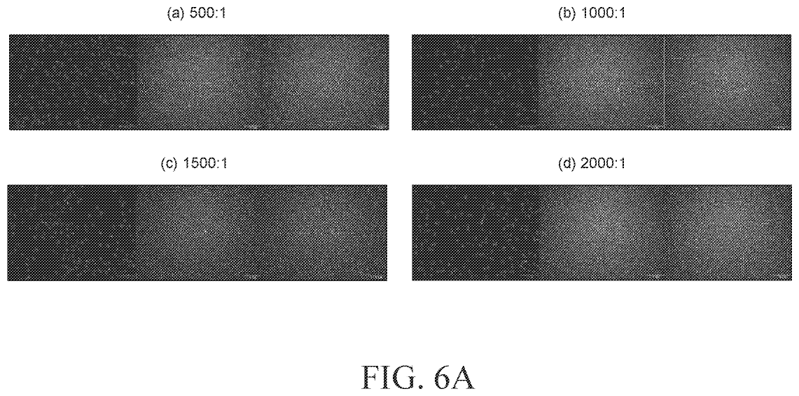

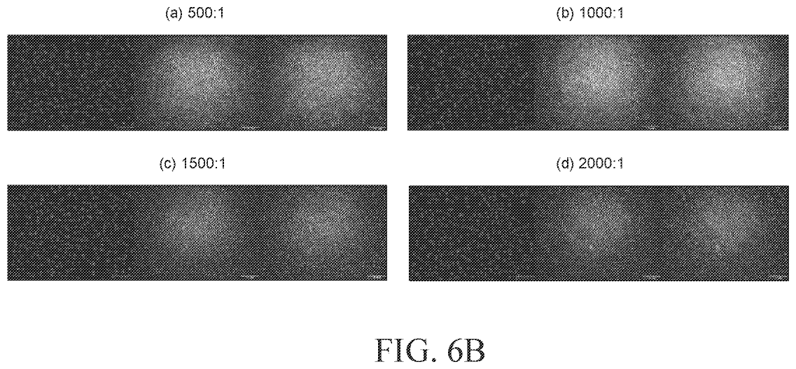

Hereinafter, referring to FIG. 6A and FIG. 6B, the results of evaluation of cell specificity of antibodies for purifying vascular endothelial cells according to an embodiment of the present disclosure will be described. In this connection, as an experimental group, HUVEC cells as human vascular endothelial cells were used as vascular endothelial cells. As a negative control, fibroblasts were used. In this connection, the content of the antibodies in accordance with the present disclosure, that is, 3.86 mg/ml concentration of the antibodies of 10D6 as described above in Example 3 was diluted in each of ratios of 500:1, 1000:1, 1500:1 and 2000:1. Then, the diluted antibodies were applied to fibroblast cells and human vascular endothelial cells respectively.

FIG. 6A and FIG. 6B show the results of evaluation of cell specificity using immunofluorescence staining process for antibodies obtained by the preparation method of the antibody against CDH5 for purifying the vascular endothelial cells according to an embodiment of the present disclosure.

First, referring to (a), (b), (c) and (d) of FIG. 6A, the results of the application of the antibodies in accordance with the present disclosure as diluted in ratios of 500:1, 1000:1, 1500:1 and 2000:1 to the fibroblasts as the negative control are shown. More specifically, it is observed that fibroblasts emit blue light. However, as the fibroblasts has no specific binding to the antibody in accordance with the present disclosure, the antibody emitting green light is not observed.

In one example, referring to (a), (b), (c) and (d) of FIG. 6B, together with blue luminescent human vascular endothelial cells, a green luminescent antibody appears on the surface of the endothelial cell. In this connection, the antibody in accordance with the present disclosure specifically binds to CDH5 on the surface of human vascular endothelial cells, allowing the detection of the human vascular endothelial cells. In particular, referring to (d) of FIG. 6B, the antibody in accordance with the present disclosure at a diluted state at 2000:1 allows a high level of detection of vascular endothelial cells. Thus, the antibody in accordance with the present disclosure has a high specificity to the vascular endothelial cells even at low concentrations. Even at low concentrations of the antibodies, the vascular endothelial cells may be detected efficiently. Furthermore, the antibody in accordance with the present disclosure may facilitate the acquisition of the endothelial cells at the high purity.

In particular, the antibody of 10D6 has the specificity to the vascular endothelial cells and has a high degree of binding to the vascular endothelial cells at a concentration of about 0.00193 mg/ml following a dilution at 2000:1.

From a result of Example 4, the antibody for purifying vascular endothelial cells, which are used in various embodiments of the present disclosure may be used to isolate high purity endothelial cells as the antibody binds specifically to the vascular endothelial cells.

Thus, the vascular endothelial cells isolated using the antibody for purifying the vascular endothelial cells in accordance with the present disclosure may have a high purity and thus may be stably clinically applied as an effective cell therapy for the prevention or treatment of cardiovascular vessel diseases.

More specifically, the antibodies used in the various embodiments of the present disclosure may purify, in a high purity. the vascular endothelial cells that may be applied directly to ischemic tissue. Thus, the antibodies may constitute a cell therapeutic composition for treatment of diseases requiring blood vessel generation, such as ischemic heart blood vessel disease, brain blood vessel disease, diabetic complications, and wound care.

Although the embodiments of the present disclosure have been described in more detail with reference to the accompanying drawings, the present disclosure is not necessarily limited to these embodiments. Many changes may be made without departing from the spirit of the present disclosure. Accordingly, the embodiments as disclosed in the present disclosure are not intended to limit the technical spirit of the present disclosure, but to describe the present disclosure. However, the scope of the technical idea of the present disclosure is not limited to these embodiments. Therefore, it should be understood that the embodiments as described above are exemplary in all respects and not restrictive. The scope of protection of the present disclosure should be interpreted by the following claims. All technical ideas falling within the equivalent scope should be interpreted as being included in the scope of the present disclosure.

SEQUENCE LISTING FREE TEXT
<110> INDUSTRY-ACADEMIC COOPERATION FOUNDATION,
YONSEI UNIVERSITY
<120> ANTIBODY FOR PURIFYING VASCULAR ENDOTHELIAL
CELL AND METHOD FOR
MAKING THEREOF
<130> 19PD5340PCT
<160> 2
<170> KoPatentIn 3.0
<210> 1
<211> 574
<212> PRT
<213> Artificial Sequence
<220>
<223> Artificial
<400> 1
Gln Glu Asn Ser Phe Thr Ile Asn Ser Val Asp Met Lys Ser Leu Pro
Asp Trp Thr Val Gln Asn Gly Lys Asn Leu Thr Leu Gln Cys Phe Ala
Asp Val Ser Thr Thr Ser His Val Lys Pro Gln His Gln Met Leu Phe
Tyr Lys Asp Asp Val Leu Phe Tyr Asn Ile Ser Ser Met Lys Ser Thr
Glu Ser Tyr Phe Ile Pro Glu Val Arg Ile Tyr Asp Ser Gly Thr Tyr
Lys Cys Thr Val Ile Val Asn Asn Lys Glu Lys Thr Thr Ala Glu Tyr
Gln Leu Leu Val Glu Gly Val Pro Ser Pro Arg Val Thr Leu Asp Lys
Lys Glu Ala Ile Gln Gly Gly Ile Val Arg Val Asn Cys Ser Val Pro
Glu Glu Lys Ala Pro Ile His Phe Thr Ile Glu Lys Leu Glu Leu Asn
Glu Lys Met Val Lys Leu Lys Arg Glu Lys Asn Ser Arg Asp Gln Asn
Phe Val Ile Leu Glu Phe Pro Val Glu Glu Gln Asp Arg Val Leu Ser
Phe Arg Cys Gln Ala Arg Ile Ile Ser Gly Ile His Met Gln Thr Ser
Glu Ser Thr Lys Ser Glu Leu Val Thr Val Thr Glu Ser Phe Ser Thr
Pro Lys Phe His Ile Ser Pro Thr Gly Met Ile Met Glu Gly Ala Gln
Leu His Ile Lys Cys Thr Ile Gln Val Thr His Leu Ala Gln Glu Phe
Pro Glu Ile Ile Ile Gln Lys Asp Lys Ala Ile Val Ala His Asn Arg
His Gly Asn Lys Ala Val Tyr Ser Val Met Ala Met Val Glu His Ser
Gly Asn Tyr Thr Cys Lys Val Glu Ser Ser Arg Ile Ser Lys Val Ser
Ser Ile Val Val Asn Ile Thr Glu Leu Phe Ser Lys Pro Glu Leu Glu
Ser Ser Phe Thr His Leu Asp Gln Gly Glu Arg Leu Asn Leu Ser Cys
Ser Ile Pro Gly Ala Pro Pro Ala Asn Phe Thr Ile Gln Lys Glu Asp
Thr Ile Val Ser Gln Thr Gln Asp Phe Thr Lys Ile Ala Ser Lys Ser
Asp Ser Gly Thr Tyr Ile Cys Thr Ala Gly Ile Asp Lys Val Val Lys
Lys Ser Asn Thr Val Gln Ile Val Val Cys Glu Met Leu Ser Gln Pro
Arg Ile Ser Tyr Asp Ala Gln Phe Glu Val Ile Lys Gly Gln Thr Ile
Glu Val Arg Cys Glu Ser Ile Ser Gly Thr Leu Pro Ile Ser Tyr Gln
Leu Leu Lys Thr Ser Lys Val Leu Glu Asn Ser Thr Lys Asn Ser Asn
Asp Pro Ala Val Phe Lys Asp Asn Pro Thr Glu Asp Val Glu Tyr Gln
Cys Val Ala Asp Asn Cys His Ser His Ala Lys Met Leu Ser Glu Val
Leu Arg Val Lys Val Ile Ala Pro Val Asp Glu Val Gln Ile Ser Ile
Leu Ser Ser Lys Val Val Glu Ser Gly Glu Asp Ile Val Leu Gln Cys
Ala Val Asn Glu Gly Ser Gly Pro Ile Thr Tyr Lys Phe Tyr Arg Glu
Lys Glu Gly Lys Pro Glu Tyr Gln Met Thr Ser Asn Ala Thr Gln Ala
Phe Trp Thr Lys Gln Lys Ala Ser Lys Glu Gln Glu Gly Glu Tyr Tyr
Cys Thr Ala Phe Asn Arg Ala Asn His Ala Ser Ser Val Pro Arg Ser
Lys Ile Leu Thr Val Arg Val Ile Leu Ala Pro Trp Lys Lys
<210> 2
<211> 552
<212> PRT
<213> Artificial Sequence
<220>
<223> Artificial
<400> 2
Asp Trp Ile Trp Asn Gln Met His Ile Asp Glu Glu Lys Asn Thr Ser
Leu Pro His His Val Gly Lys Ile Lys Ser Ser Val Ser Arg Lys Asn
Ala Lys Tyr Leu Leu Lys Gly Glu Tyr Val Gly Lys Val Phe Arg Val
Asp Ala Glu Thr Gly Asp Val Phe Ala Ile Glu Arg Leu Asp Arg Glu
Asn Ile Ser Glu Tyr His Leu Thr Ala Val Ile Val Asp Lys Asp Thr
Gly Glu Asn Leu Glu Thr Pro Ser Ser Phe Thr Ile Lys Val His Asp
Val Asn Asp Asn Trp Pro Val Phe Thr His Arg Leu Phe Asn Ala Ser
Val Pro Glu Ser Ser Ala Val Gly Thr Ser Val Ile Ser Val Thr Ala
Val Asp Ala Asp Asp Pro Thr Val Gly Asp His Ala Ser Val Met Tyr
Gln Ile Leu Lys Gly Lys Glu Tyr Phe Ala Ile Asp Asn Ser Gly Arg
Ile Ile Thr Ile Thr Lys Ser Leu Asp Arg Glu Lys Gln Ala Arg Tyr
Glu Ile Val Val Glu Ala Arg Asp Ala Gln Gly Leu Arg Gly Asp Ser
Gly Thr Ala Thr Val Leu Val Thr Leu Gln Asp Ile Asn Asp Asn Phe
Pro Phe Phe Thr Gln Thr Lys Tyr Thr Phe Val Val Pro Glu Asp Thr
Arg Val Gly Thr Ser Val Gly Ser Leu Phe Val Glu Asp Pro Asp Glu
Pro Gln Asn Arg Met Thr Lys Tyr Ser Ile Leu Arg Gly Asp Tyr Gln
Asp Ala Phe Thr Ile Glu Thr Asn Pro Ala His Asn Glu Gly Ile Ile
Lys Pro Met Lys Pro Leu Asp Tyr Glu Tyr Ile Gln Gln Tyr Ser Phe
Ile Val Glu Ala Thr Asp Pro Thr Ile Asp Leu Arg Tyr Met Ser Pro
Pro Ala Gly Asn Arg Gln Gln Val Ile Ile Asn Ile Thr Asp Val Asp
Glu Pro Pro Ile Phe Gln Gln Pro Phe Tyr His Phe Gln Leu Lys Glu
Asn Gln Lys Lys Pro Leu Ile Gly Thr Val Leu Ala Met Asp Pro Asp
Ala Ala Arg His Ser Ile Gly Tyr Ser Ile Arg Arg Thr Ser Asp Lys
Gly Gln Phe Phe Arg Val Thr Lys Lys Gly Asp Ile Tyr Asn Glu Lys
Glu Leu Asp Arg Glu Val Tyr Pro Trp Tyr Asn Leu Thr Val Glu Ala
Lys Glu Leu Asp Ser Thr Gly Thr Pro Thr Gly Lys Glu Ser Ile Val
Gln Val His Ile Glu Val Leu Asp Glu Asn Asp Asn Ala Pro Glu Phe
Ala Lys Pro Tyr Gln Pro Lys Val Cys Glu Asn Ala Val His Gly Gln
Leu Val Leu Gln Ile Ser Ala Ile Asp Lys Asp Ile Thr Pro Arg Asn
Val Lys Phe Lys Phe Ile Leu Asn Thr Glu Asn Asn Phe Thr Leu Thr
Asp Asn His Asp Asn Thr Ala Asn Ile Thr Val Lys Tyr Gly Gln Phe
Asp Arg Glu His Thr Lys Val His Phe Leu Pro Val Val Ile Ser Asp
Asn Gly Met Pro Ser Arg Thr Gly Thr Ser Thr Leu Thr Val Ala Val
Cys Lys Cys Asn Glu Gln Gly Glu Phe Thr Phe Cys Glu Asp Met Ala
Ala Gln Val Gly Val Ser Ile Gln

[National R&D projects that supported this invention]

[Assignment unique number] HI16C2211

[Department name] Ministry of Health and Welfare

[Research Management Professional Institution] Korea Health Industry Development Institute

[Research Project Name] Stem cells, regenerative medicine commercialization/performance-creating mediation research

[Research Study Name] Development of cell therapy for peripheral arterial disease using vascular endothelial cells differentiated from induced pluripotent stem cells [Contribution rate] 4/10

[Organizing Agency] Yonsei University Industry-Academic Cooperation Foundation

[Study period] 20161108˜20210731

[National R&D projects that supported this invention]

[Assignment unique number] HI15C2782

[Department name] Ministry of Health and Welfare

[Research Management Professional Institution] Korea Health Industry Development Institute

[Research Project Name] Development of advanced medical technology/stem cell, international cooperation in establishing a foundation for regenerative medicine

[Research Study Name] Development of direct cell conversion method to vascular endothelial cells using nanoparticles

[Contribution rate] 3/10

[Organizing Agency] Yonsei University Industry-Academic Cooperation Foundation

[Study period] 20151201˜20191130

[National R&D projects that supported this invention]

[Assignment unique number] 2015M3A9C6031514

[Department name] Ministry of Science and ICT

[Research Management Professional Institution] National Research Foundation of Korea

[Research Project Name] Biomedical technology development business

[Research Study Name] Differentiation and separation of induced pluripotent stem cells into subtypes of cardiomyocytes, and development of technology based on cell therapy and drug evaluation using the same

[Contribution rate] 3/10

[Organizing Agency] Yonsei University Industry-Academic Cooperation Foundation

[Study period] 20150601˜20200531

1. An antibody for purifying a vascular endothelial cell, the antibody containing a heavy-chain variable domain and a light-chain variable domain specifically binding to an extracellular matrix domain of PECAM1 having an amino acid sequence represented by SEQ ID NO: 1 or CDH5 having an amino acid sequence represented by SEQ ID NO: 2.

2. The antibody of claim 1, wherein the antibody includes a monoclonal antibody specifically binding to the extracellular matrix domain of PECAM1 or the extracellular matrix domain of CDH5.

3. The antibody of claim 1, wherein the antibody further includes a heavy-chain invariable domain and a magnetic particle,
wherein the magnetic particle is attached to the heavy-chain invariable domain of the antibody.

4. A method for producing an antibody for purifying a vascular endothelial cell, the method comprising:
injecting a protein containing an extracellular matrix domain of PECAM1 having an amino acid sequence represented by SEQ ID NO: 1 into an antibody producing subject so as to produce an antibody against the PECAM1;
determining a positive clone reacting with an antigen of the PECAM1 from the antibody producing subject; and
separating the antibody against the PECAM1 from the positive clone.

5. The method of claim 4, wherein the method further includes, prior to the injection of the protein into the antibody producing subject:
preparing a recombinant plasmid vector containing a base sequence expressing the extracellular matrix domain of the PECAM1;
transfecting the recombinant plasmid vector into a host cell; and
obtaining the protein containing the extracellular matrix domain of the PECAM1.

6. The method of claim 4, wherein the determining of a positive clone includes:
separating a plurality of B lymphocytes from the antibody producing subject;
fusing the plurality of B lymphocytes with myeloma cells respectively to produce fused cells;
culturing the plurality of the fused cells respectively to form a clone; and
determining the positive clone to react with the antigen of the PECAM1 from the formed clone,
wherein separating the antibody includes purifying a monoclonal antibody against the PECAM1 from the positive clone.

7. The method of claim 4, wherein a concentration of the separated antibodies against the PECAM1 is at least 3.5 mg/ml.

8. The method of claim 4, wherein the separated antibodies against the PECAM1 maintain a specificity and a binding affinity to the extracellular matrix domain of the PECAM1 at a concentration of 0.004 mg/ml or lower of the antibodies.

9. A method for producing an antibody for purifying a vascular endothelial cell, the method comprising:
injecting a protein containing an extracellular matrix domain of CDH5 having an amino acid sequence represented by SEQ ID NO: 2 into an antibody producing subject so as to produce an antibody against the CDH5;
determining a positive clone reacting with an antigen of the CDH5 from the antibody producing subject; and
separating the antibody against the CDH5 from the positive clone.

10. The method of claim 9, wherein the method further includes, prior to the injection of the protein into the antibody producing subject:
preparing a recombinant plasmid vector containing a base sequence expressing the extracellular matrix domain of CDH5;
transfecting the recombinant plasmid vector to a host cell; and
obtaining the protein containing the extracellular matrix domain of CDH5.

11. The method of claim 9, wherein the determining of the positive clone includes:
separating a plurality of B lymphocytes from the antibody producing subject;
fusing the plurality of B lymphocytes with myeloma cells respectively to produce fused cells;
culturing the plurality of the fused cells respectively to form a clone; and
determining the positive clone to react with the antigen of CDH5 from the formed clone,
wherein separating the antibody includes purifying a monoclonal antibody against the CDH5 from the positive clone.

12. The method of claim 9, wherein a concentration of the separated antibodies against the CDH5 is at least 3.5 mg/ml.

13. The method of claim 9, wherein the separated antibodies against the CDH5 maintain a specificity and a binding affinity to the extracellular matrix domain of the CDH5 at a concentration of 0.004 mg/ml or lower of the antibodies.

14. A method for purifying a vascular endothelial cell, the method comprising:
differentiating an endothelial cell from a stem cell to obtain a cell cluster containing a vascular endothelial cell and an endothelial cell different from the vascular endothelial cell;
applying the antibody of one of claims 1 to 3 to the cell cluster, such that a protein of PECMA1 or a protein of CDH5 present on a surface of the vascular endothelial cell immuno-responses to the antibody for purifying the vascular endothelial cell; and
sorting a cell exhibiting fluorescence in an immunofluorescence staining process due to the immuno-response as a vascular endothelial cell.

15. The method of claim 14, wherein the stem cell includes a human pluripotent stem cell or a human induced pluripotent stem cell.

16. The method of claim 14, wherein the differentiating includes culturing the stem cell in a DLL4-treated medium to differentiate the endothelial cell.

17. The method of claim 14, wherein the antibody for purifying the vascular endothelial cell further contains a magnetic particle,
wherein the sorting includes:
permeating the antibody-treated cell cluster into a column, wherein the column receives a metallic particle therein, and a magnetic force is externally applied to the column; and
sorting a cell bound to the metallic particle located inside the column as the vascular endothelial cell.

18. The method of claim 17, wherein the sorting includes sorting the vascular endothelial cell by blocking the magnetic force externally applied to the column.

19. A kit for purifying a vascular endothelial cell, the kit comprising the antibody of one of claims 1 to 3.


SEQUENCE LISTING (2 sequences)

SEQ ID NO: 1 - protein, 574 aa, Artificial Sequence
  Synthesis sequence binding to extracellular matrix domain of PECAM1 (platelet endothelial cell adhesi on molecule1)
QENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPEYQMTSNATQAFWTKQKASKEQEGEYYCTAFNRANHASSVPRSKILTVRVILAPWKK

SEQ ID NO: 2 - protein, 552 aa, Artificial Sequence
  Synthesis sequence binding to extracellular matrix domain of CDH5 (cadherin-5)
DWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRQQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFILNTENNFTLTDNHDNTANITVKYGQFDREHTKVHFLPVVISDNGMPSRTGTSTLTVAVCKCNEQGEFTFCEDMAAQVGVSIQ